(19) United States
(12) Patent Application Publication
(10) Pub. No.: US 2024/0386551 A1
(43) Pub. Date: Nov. 21, 2024

(54) SYSTEM AND METHOD FOR SEGMENTING MEDICAL IMAGES

(71) Applicants: Nanyang Technological University, Singapore (SG); Singapore Health Services Pte Ltd, Singapore (SG)

(72) Inventors: Jagath Chandana RAJAPAKSE, Singapore (SG); Abhinandan PADHI, Singapore (SG); Vivek ADRAKATTI, Singapore (SG); Chun Hung HOW, Singapore (SG); Yi Hao CHAN, Singapore (SG); Iram Rais Alam KHAN, Singapore (SG); Choie Cheio Tchoyoson LIM, Singapore (SG)

(21) Appl. No.: 18/665,536

(22) Filed: May 15, 2024

(30) Foreign Application Priority Data
May 15, 2023 (SG) 10202301347W

(51) Int. Cl. G06T 7/00; G06T 7/11; G06V 10/25; G16H 30/40; G16H 50/20

(52) U.S. Cl. CPC G06T 7/0012; G06T 7/11; G06V 10/25; G16H 30/40; G16H 50/20; G06T 2207/30016; G06T 2207/30096

(57) ABSTRACT

The present disclosure generally relates to a computer system and a computerized method for segmenting a medical image of an organ. The method comprises: detecting, using an object detection model, a ROI in the medical image, the ROI comprising a lesion in the organ; demarcating, using the object detection model, a bounding box around the ROI; extracting a localized image comprising the ROI from the medical image, the localized image defined by the bounding box; segmenting, using an image segmentation model that is independent from the object detection model, the localized image comprising the ROI; predicting, using the image segmentation model, a segmentation mask of the lesion in the localized image; and outputting the segmentation mask from the localized image to the medical image to facilitate medical diagnosis of the lesion, wherein the object detection model is trained using a first dataset of training images comprising medical images of the organ, each medical image in the first dataset comprising a ground-truth bounding box for one or more lesions in the organ.

### CROSS REFERENCE TO RELATED APPLICATION(S)

The present disclosure claims the benefit of Singapore Patent Application No. 10202301347W filed on 15 May 2023, which is incorporated in its entirety by reference herein.

### TECHNICAL FIELD

The present disclosure generally relates to a system and method for segmenting medical images. More particularly, the present disclosure describes various embodiments of a computer system and a computerized method for segmenting medical images such as computed tomography scans for diagnosing lesions such as intracranial haemorrhages.

### BACKGROUND

Lesions are regions in human organs or tissue that are damaged because of injury and/or disease. One example of lesions is intracranial haemorrhage (ICH) which refers to the extravascular accumulation of blood within different intracranial spaces. The causes of ICH are diverse, such as head trauma or various medical conditions including hypertension and aneurysms. ICH is usually an emergency and in serious cases, it can lead to permanent neurological damage or even death. ICH is a life-threatening condition as the 30-day mortality rate for ICH ranges from about 35% to 52%, where only 20% of survivors are expected to have full functional recovery within 6 months. ICH is also a relatively common condition—the frequency of acute ICH worldwide is about 24.6 per 100,000 persons per year.

Computed tomography (CT) is a common imaging modality to diagnose ICH and other neurological diseases. To accurately locate and identify the type of ICH, CT scans of the brain are examined by neuroradiologists. However, this process can be challenging and time-consuming. While acute blood appears hyperdense or white on the CT scan and should not cause much difficulty in diagnosis, ICH detection and interpretation may pose some challenges to junior radiologists, especially if the bleeding amount is small. Diagnosis of ICH may be delayed in busy hospitals where there is increased utilization of CT machines. Solutions are thus needed for quicker and more accurate diagnosis of ICH so that clinicians can intervene timely.

Therefore, in order to address or alleviate at least one of the aforementioned problems and/or disadvantages, there is a need to provide an improved system and method for diagnosis of ICH or lesions from medical images.

### SUMMARY

According to a first aspect of the present disclosure, there is a computerized method for segmenting a medical image of an organ. The method comprises: detecting, using an object detection model, a region of interest in the medical image, the region of interest comprising a lesion in the organ; demarcating, using the object detection model, a bounding box around the region of interest; extracting a localized image comprising the region of interest from the medical image, the localized image defined by the bounding box; segmenting, using an image segmentation model that is independent from the object detection model, the localized image comprising the region of interest; predicting, using the image segmentation model, a segmentation mask of the lesion in the localized image; and outputting the segmentation mask from the localized image to the medical image to facilitate medical diagnosis of the lesion, wherein the object detection model is trained using a first dataset of medical images of the organ, the first dataset of medical images comprising ground-truth bounding boxes for lesions.

According to a second aspect of the present disclosure, there is a computer system for segmenting a medical image of an organ. The system comprises: an object detection model that is trained using a first dataset of medical images of the organ, the first dataset of medical images comprising ground-truth bounding boxes for lesions; an image segmentation model that is independent from the object detection model; and a processor. The processor is configured for: detecting, using the object detection model, a region of interest in the medical image, the region of interest comprising a lesion in the organ; demarcating, using the object detection model, a bounding box around the region of interest; extracting a localized image comprising the region of interest from the medical image, the localized image defined by the bounding box; segmenting, using the image segmentation model, the localized image comprising the region of interest; predicting, using the image segmentation model, a segmentation mask of the lesion in the localized image; and outputting the segmentation mask from the localized image to the medical image to facilitate medical diagnosis of the lesion.

A computer system and a computerized method for segmenting medical images according to the present disclosure are thus disclosed herein. Various features and advantages of the present disclosure will become more apparent from the following detailed description of the embodiments of the present disclosure, by way of non-limiting examples only, along with the accompanying drawings.

### BRIEF DESCRIPTION OF THE DRAWINGS

FIG. 1A and FIG. 1B are illustrations of a computer system for segmenting medical images, according to embodiments of the present disclosure.

FIG. 2A and FIG. 2B are flowchart illustrations of a computerized method for segmenting medical images, according to embodiments of the present disclosure.

FIG. 3A to FIG. 3C are illustrations of segmentation results from the computer system and computerized method.

### DETAILED DESCRIPTION

For purposes of brevity and clarity, descriptions of embodiments of the present disclosure are directed to a computer system and a computerized method for segmenting medical images, in accordance with the drawings. While parts of the present disclosure will be described in conjunction with the embodiments provided herein, it will be understood that they are not intended to limit the present disclosure to these embodiments. On the contrary, the present disclosure is intended to cover alternatives, modifications and equivalents to the embodiments described herein, which are included within the scope of the present disclosure as defined by the appended claims. Furthermore, in the following detailed description, specific details are set forth in order to provide a thorough understanding of the present disclosure. However, it will be recognized by an individual having ordinary skill in the art, i.e. a skilled person, that the present disclosure may be practiced without specific details, and/or with multiple details arising from combinations of features of particular embodiments. In a number of instances, well-known systems, methods, procedures, and components have not been described in detail so as to not unnecessarily obscure features of the embodiments of the present disclosure.

In embodiments of the present disclosure, depiction of a given element or consideration or use of a particular element number in a particular figure or a reference thereto in corresponding descriptive material can encompass the same, an equivalent, or an analogous element or element number identified in another figure or descriptive material associated therewith.

References to “an embodiment/example”, “another embodiment/example”, “some embodiments/examples”, “some other embodiments/examples”, and so on, indicate that the embodiment(s)/example(s) so described may include a particular feature, structure, characteristic, property, element, or limitation, but that not every embodiment/example necessarily includes that particular feature, structure, characteristic, property, element or limitation. Furthermore, repeated use of the phrase “in an embodiment/example” or “in another embodiment/example” does not necessarily refer to the same embodiment/example.

The terms “comprising”, “including”, “having”, and the like do not exclude the presence of other features/elements/steps than those listed in an embodiment. Recitation of certain features/elements/steps in mutually different embodiments does not indicate that a combination of these features/elements/steps cannot be used in an embodiment.

As used herein, the terms “a” and “an” are defined as one or more than one. The use of “/” in a figure or associated text is understood to mean “and/or” unless otherwise indicated. The term “set” is defined as a non-empty finite organization of elements that mathematically exhibits a cardinality of at least one (e.g. a set as defined herein can correspond to a unit, singlet, or single-element set, or a multiple-element set), in accordance with known mathematical definitions. The recitation of a particular numerical value or value range herein is understood to include or be a recitation of an approximate numerical value or value range.

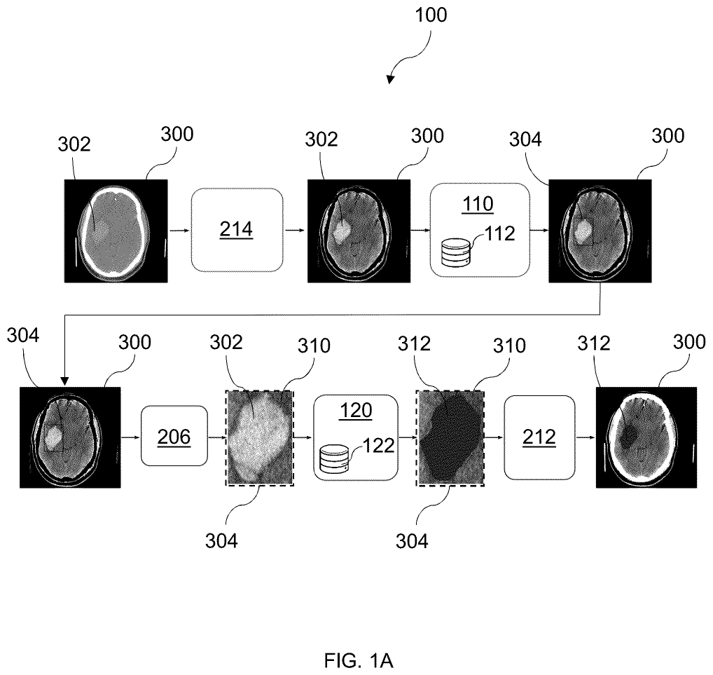

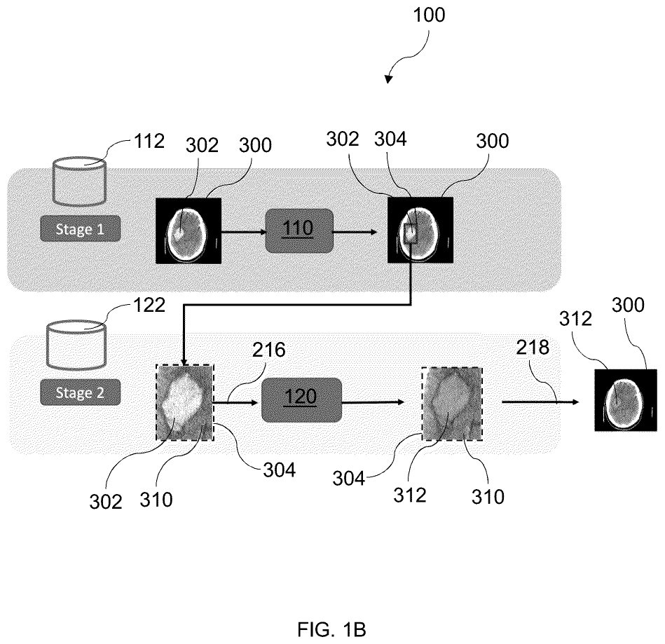

Representative or exemplary embodiments of the present disclosure describe a computer system 100 and computerized method 200 for segmenting medical images 300. Specifically, the system 100 and method 200 segments a medical image 300 of an organ, such as a CT scan of a brain, for diagnosing a lesion such as an intracranial haemorrhage (ICH) in the brain. With reference to FIG. 1A and FIG. 1B, the system 100 includes an object detection model 110 and an image segmentation model 120 that is independent from the object detection model 110. The object detection model 110 and the image segmentation model 120 are machine learning models that run independently from each other to segment the medical image 300 and diagnose the lesion.

The system 100 further includes a processor configured for performing various steps of the computerized method 200 in response to non-transitory instructions operative or executed by the processor. The non-transitory instructions are stored on a memory of the computer system 100 and may be referred to as computer-readable storage media and/or non-transitory computer-readable media. Non-transitory computer-readable media include all computer-readable media, with the sole exception being a transitory propagating signal per se.

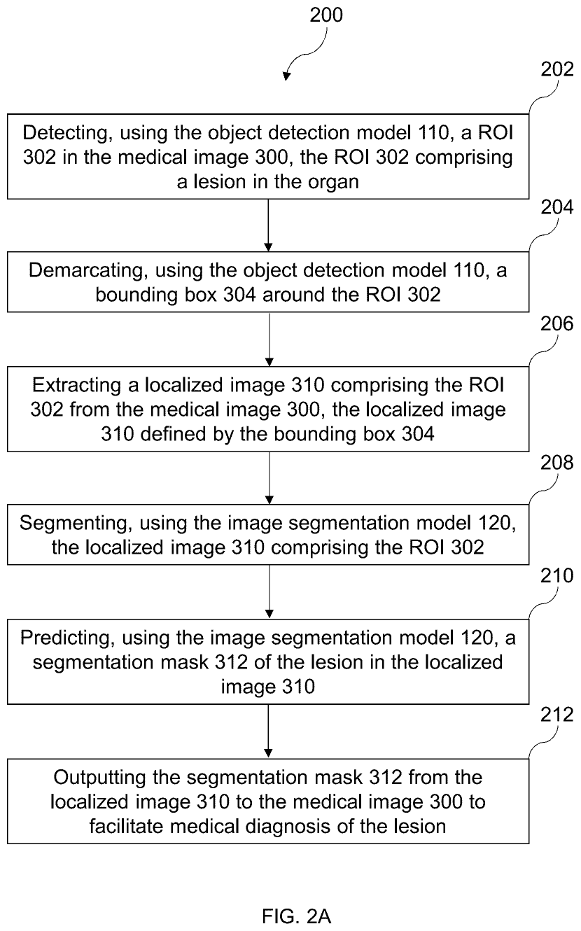

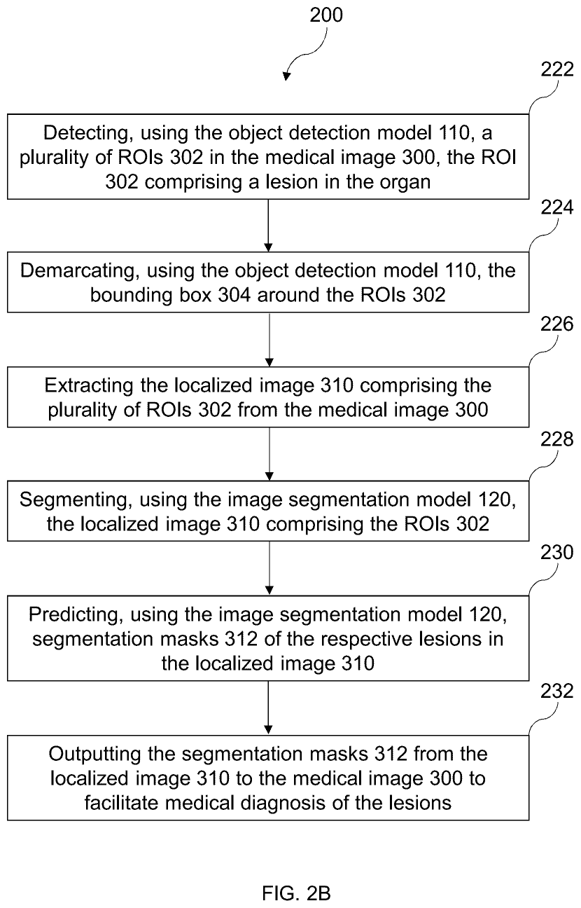

As shown in FIG. 1A and FIG. 1B, the system 100 and method 200 perform machine learning inference in two stages—the first stage performed by the object detection model 110 and the second stage performed by the image segmentation model 120. Further with reference to FIG. 2A, in the first stage, the method 200 includes a step 202 of detecting, using the object detection model 110, a region of interest (ROI) 302 in the medical image 300, the ROI 302 comprising a lesion in the organ. The method 200 includes a step 204 of demarcating, using the object detection model 110, a bounding box 304 around the ROI 302.

In the second stage, the method 200 includes a step 206 of extracting a localized image 310 comprising the ROI 302 from the medical image 300, the localized image 310 defined by the bounding box 304. The method 200 includes a step 208 of segmenting, using the image segmentation model 120, the localized image 310 comprising the ROI 302. The method 200 includes a step 210 of predicting, using the image segmentation model 120, a segmentation mask 312 of the lesion in the localized image 310. The method 200 includes a step 212 of outputting the segmentation mask 312 from the localized image 310 to the medical image 300 to facilitate medical diagnosis of the lesion.

In some embodiments, the organ may have a plurality of lesions. With reference to FIG. 2B, in the first stage, the method 200 includes a step 222 of detecting, using the object detection model 110, a plurality of ROIs 302 in the medical image 300, each ROI 302 comprising a lesion in the organ. The method 200 includes a step 224 of demarcating, using the object detection model 110, the bounding box 304 around the plurality of ROIs 302. Specifically, object detection model 110 detects multiple bounding sub-boxes for the multiple ROIs 302, and demarcates a single bounding box 304 around all the sub-boxes. The bounding box 304 is thus the smallest possible box that would encompass all the detected sub-boxes, regardless of the subtype of the lesion in each respective sub-box. Since the bounding box 304 is always rectangular, the bounding box 304 would be formed by the overall minimum and maximum values of the coordinates of all the sub-boxes' corners. Unionizing all the smaller sub-boxes within the single bounding box 304 advantageously improves computational efficiency and segmentation accuracy in the second stage, especially when GPU parallel computing is not available.

In the second stage, the method 200 includes a step 226 of extracting the localized image 310 comprising the plurality of ROIs 302 from the medical image 300. Notably, the localized image 310 defined by the bounding box 304 such that all the ROIs 302 are contained within the single bounding box 304. The method 200 includes a step 228 of segmenting, using the image segmentation model 120, the localized image 310 comprising the plurality of ROIs 302. The method 200 includes a step 230 of predicting, using the image segmentation model 120, segmentation masks 312 of the respective lesions in the localized image 310. Notably, the localized image 310 is defined by the bounding box 304 such that all the ROIs 302, each ROI 302 comprising a respective lesion, are contained within the single bounding box 304. The method 200 includes a step 232 of outputting the segmentation masks 312 from the localized image 310 to the medical image 300 to facilitate medical diagnosis of the lesions.

In some embodiments, the method 200 may include a step 214 of pre-processing the medical image 300 before the step 202 or 222 of detecting the ROIs 302. The pre-processing may include windowing the medical image 300. For example, the medical image 300 is a 2D slice from a CT scan in the DICOM (Digital Imaging and Communications in Medicine) format. The CT slice is converted to a 512×512 pixel array and the pixel intensities are represented in dimensionless Hounsfield Units (HU). The HU scale ranges from −1024 HU to +3071 HU, where −1000 HU represents the density of air, and +2000 HU represents the density of bone. However, the default HU range usually provides poor contrast between normal bone tissue and lesion tissue.

Windowing the CT slice adjusts the contrast of the CT slice. In this windowing process, which is also referred to as contrast stretching, the window width and window level properties of the CT slice are adjusted. Ideal window width and window level values were pre-obtained from DICOM metadata for the object detection model 110. After windowing, the pixel intensities are scaled to the range of 0 to 255 for use with the object detection model 110.

Alternatively or additionally, the pre-processing may include removing regions of normal organ tissue and calcification. For example, the organ is a brain, and the pre-processing includes removing regions of skull tissue and calcification in the CT slice. Skull tissue and calcification in the brain appear as bright white spots on the CT slice due to their high density, though both are just normal or benign tissue in the brain. However, ICH regions also appear white on the CT slice, albeit slightly dimmer. This can result in false positives for the skull and calcification pixels, especially if the ICH is located adjacent or near to the skull or calcification regions. To address this problem, pixels with intensity greater than a preset threshold for the CT slice are removed, which would remove most of the skull and calcification pixels. The pre-processing thus removes the skull and calcification regions in the CT slice, thereby improving the detection and segmentation accuracy of the machine learning models 110,120.

The object detection model 110 is a trained machine learning model that is trained using a first dataset 112 of training images. The first dataset 112 of training images comprises medical images 300 of the organ, wherein each medical image 300 in the first dataset 112 comprises a ground-truth bounding box for one of more lesions in the organ. For example, the object detection model 110 includes a YOLOv5 model. The image segmentation model 120 may be a trained or untrained machine learning model. For example, the image segmentation model 120 includes an untrained Expectation-Maximization (EM) algorithm. For example, the image segmentation model 120 is a trained machine learning model that is trained, independently from the training of the object detection model 110, using a second dataset 122 of training images. The second dataset 122 of training images comprises localized images 310 of lesions in the organ, wherein each localized image 310 in the second dataset 122 comprises one or more ground-truth segmentation masks for one or more lesions in the organ. For example, the trained image segmentation model 120 includes a TransDeepLab model.

In some embodiments, the method 200 includes steps of training the object detection model 110 and/or the image segmentation model 120. The training steps may optionally include performing data augmentation on the respective datasets 112,122 of medical images 300. The data augmentation may include, but is not limited to, augmentation of hue properties, augmentation of saturation properties, augmentation of value properties, translation of the medical images 300, scaling of the medical images 300, random flipping (horizontally and/or vertically) of the medical images 300, random rotation of the medical images 300, and creation of mosaics of the medical images 300. The data augmentation increases the size of the datasets 112,122 and enables more effective training of the machine learning models 110,120.

Preferably, the object detection model 110 includes the YOLOv5 model. YOLOv5 is a model in the You Only Look Once (YOLO) family of computer vision models designed for the task of object detection. The YOLOv5 model comes in five variants-nano, small, medium, large, and extra-large. Each variant has a different number of trainable parameters and requires a different amount of time to train. The choice of a variant of the YOLOv5 model depends on the hardware constraints and desired trade-off between detection speed and accuracy. In embodiments of the present disclosure for detecting lesions, based on the hardware constraints and the high accuracy required, the large variant was chosen for this task. Further, hyperparameters of the YOLOv5 model are optimized using a Genetic Algorithm to improve the ICH detection accuracy. In the first stage, the 512×512 pre-processed CT slices are inputted to the YOLOv5 model to detect the ROIs 302 with lesions and to demarcate the ROIs 302 with the bounding boxes 304. Specifically, the YOLOv5 model uses bounding box regression to draw the bounding boxes 304 around the ROIs 302.

In some embodiments as shown in FIG. 1A, the image segmentation model 120 includes the untrained EM algorithm. The EM algorithm is an unsupervised algorithm that is used to find local maximum likelihood parameters of a statistical model. The EM algorithm can be used for image segmentation due to the distinct pixel intensity distributions of the various tissue types in a brain CT scan. Moreover, since the EM algorithm does not require training, it overcomes the problem of training data with ground-truth segmentation masks.

In the second stage, the EM algorithm is used for unsupervised segmentation of ICH detected in the CT slices from the first stage. ICH segmentation using the EM algorithm is possible as there are three types of brain tissues, each of which is characterized by distinct pixel intensities in a CT slice-normal brain matter (dark grey), ICH lesions (medium grey to dim white), and skull/calcification tissue (bright white). The collection of pixel intensities for each type of brain tissue can be characterized by a Gaussian distribution. A CT slice can have a mixture of three such Gaussian distributions, where the resulting group of pixel intensities is called a Gaussian Mixture Model (GMM). Due to the presence of the mixture of Gaussian distributions in the CT slices, the EM algorithm is able to perform image segmentation effectively.

The EM algorithm has fewer hyperparameters compared to the YOLOv5 model. For segmenting lesions, the most significant hyperparameters are the number of GMM components (i.e. the number of distributions per GMM), window width, window level, and kernel size for a median filter. As mentioned above, the localized images 310 comprising the ROIs 302 are extracted and inputted to the EM algorithm, which segments the localized images 310 and predicts the segmentation masks 312. Specifically, the EM algorithm predicts the segmentation mask 312 and assigns mask pixels to the foreground, while other pixels are assigned to the background.

The number of pixel distributions present in each bounding box 304 from the YOLOv5 model is dependent on the type of ICH detected in the CT slice. For example, in CT slices with ICH lesions near the centre of the brain, there may only be two tissue types (ICH and grey matter). For example, in CT slices with ICH near the skull, three tissue types might be observed (ICH, grey matter, and background pixels). The number of GMM components was adjusted based on the type of ICH detected by the YOLOv5 model. It should be noted that removal of skull and calcification regions in the CT slice would decrease the number of GMM components by 1, thereby reducing false positives and improving segmentation accuracy.

In one embodiment, the second stage may include a step of windowing the localized image 310 before the step 208 or 228 of segmenting the localized image 310. This is because the window width and window level are important parameters for the EM algorithm. Although windowing was applied during data pre-processing of the CT slices, windowing is re-applied in the second stage for the EM algorithm using a window width of 50 HU and a window level of 50 HU. These fixed values for the window width and window level were chosen based on experiments that indicated an increase in segmentation accuracy using such a windowing configuration.

In one embodiment, the second stage may include a step of denoising the localized image 310 before the step 208 or 228 of segmenting the localized image 310. For example, the localized images 310 are denoised using a median filter, i.e. a median blur filter. A 7×7 median blur filter was applied prior to the working of the EM algorithm. The median blur filter smoothed out noise in the localized images 310, allowing the EM algorithm to predict segmentation masks 312 where the lesions have relatively smooth borders. The median blur filter was also chosen because the CT slices are relatively low-resolution (512×512), so it is especially useful in case a CT slice has noise or artifacts due to a low-quality CT scan.

The EM algorithm then outputs the segmentation masks 312 from the localized images 310 to the CT slices to facilitate medical diagnosis of the lesions. For example, the segmentation masks 312 are overlaid over the original CT slices, allowing clinicians to visually identify the ICH lesions from the segmentation masks 312.

In some embodiments as shown in FIG. 1B, the image segmentation model 120 includes the trained TransDeepLab model. The TransDeepLab model is an attention-based hybrid of U-Net and transformer architectures. It is an extension of DeepLab, which is a semantic segmentation architecture, that uses Swin Transformer blocks to encode the image and the bottleneck performs Atrous Spatial Pyramid Pooling to exploit multi-scale features from the hierarchical encoder.

The localized images 310 comprising the ROIs 302 are extracted and inputted to the TransDeepLab model, which segments the localized images 310 and predicts the segmentation masks 312. Specifically, the TransDeepLab model has a Sigmoid function as the last convolutional layer, where a localized image 310 is processed into a probability map such that each pixel in the localized image 310 is assigned with a probability of the pixel being with a lesion. The Sigmoid function has a probability threshold that can be adjusted by the user, such as 0.5. Pixels with probabilities above the threshold are assigned to the foreground, i.e. representing the lesion, and pixels with probabilities below threshold are assigned to the background, thereby predicting a binary segmentation mask 312.

Before segmentation by the TransDeepLab model, the localized images 310 may be resized in a step 216 to the training configuration of the TransDeepLab model. For example, the localized images 310 are resized to 224×224×3 by bicubic interpolation and duplicating the third channel 3 times. The output channel of the last convolutional layer of the TransDeepLab model is preferably set to 2. Image segmentation is performed by the TransDeepLab model within the resized localized images 310, thereby predicting the binary segmentation masks 312, wherein the pixels are classified as background or foreground, the foreground pixels representing the lesion.

The TransDeepLab model then outputs the segmentation masks 312 from the localized images 310 to the CT slices to facilitate medical diagnosis of the lesions. The segmentation masks 312 may be resized in a step 218 back to the original size of the localized images 310 due to the earlier resizing step 216. The resized segmentation masks 312 may then be overlaid over the original CT slices, allowing clinicians to visually identify the ICH lesions from the segmentation masks 312.

In some embodiments, the object detection model 110 may include the deep convolutional network Faster R-CNN and/or the image segmentation model 120 may include the Swin-Unet model. The Swin-Unet model has a similar structure as the U-Net architecture, but the convolutional blocks are replaced by Swin Transformer blocks and the patch merging module performs down-sampling. It will be appreciated that the various combinations of the object detection model 110 and image segmentation model 120 may be used according to their various examples described in the present disclosure.

The system 100 and method 200 uses a two-stage approach in the machine learning inference, wherein the object detection model 110 (e.g. YOLOv5 model) detects the lesions in the first stage, and the image segmentation model 120 (e.g. EM algorithm or TransDeepLab model) segments the detected lesions in the second stage.

During the second stage, the weights in the YOLOv5 model are frozen, and ICH lesions are segmented by processing only the parts of the CT slices within the bounding boxes 304, i.e. the localized images 310. Performing lesion detection in the first stage thus helps the second stage to focus on the ROIs 302 that contain the detected lesions, hence improving segmentation accuracy.

The two-stage approach leverages the ease of object detection relative to image segmentation, and achieves improved segmentation performance as ICH localization prior to segmentation helps to reduce noise and false positives. A robust and well-trained object detection model 110 can demarcate a tight bounding box 304 around the ICH lesions. Extracting the localized image 310 within the bounding box 304 and directly inputting it to the image segmentation model 120 helps to improve segmentation performance. This is because the localized image 310 is smaller than the original medical image 300, which helps the segmentation algorithm avoid noisy regions and reduce false positives during segmentation.

Various embodiments of the object detection model 110 and image segmentation model 120 were trained and evaluated using an exemplary first dataset 112 and an exemplary second dataset 122.

In one example, the object detection model 110 includes the YOLOv5 model and the first dataset 112 was created to train and evaluate the YOLOv5 model for ICH detection. The first dataset 112 was derived from the Qure AI Head CT CQ500 dataset and the Radiological Society of North America (RSNA) ICH dataset. The CQ500 dataset contains 491 head CT scans with a total of 193,317 CT slices, and the RSNA dataset contains 874,035 head CT slices. Both datasets contain an exceedingly large number of CT slices distributed over five subtypes of ICH. However, the CT slices were only annotated with radiologists' reads regarding the presence of ICH in each CT slice and the specific subtype of ICH observed. Another dataset called the Brain Haemorrhage Extended (BHX) dataset, which is an extension of the CQ500 dataset, was used to provide 39,668 ground-truth bounding boxes for the five subtypes of ICH lesions that were present in a total of 23,409 CQ500 CT slices. The BHX bounding boxes and their corresponding CQ500 CT slices were used for ICH detection experiments using the YOLOv5 model, with an 80:10:10 split for the training, validation, and test datasets. The YOLOv5 model was trained on bounding box coordinates and ICH subtype labels for 200 epochs with batch size 64, and with 29 hyperparameters as determined by the Genetic Algorithm.

As the original CQ500 and RSNA datasets lack ground-truth annotations of bounding boxes and segmentation masks, manual annotations were required. 347 CT slices distributed approximately equally over the five subtypes of ICH were randomly selected from both datasets—100 from the CQ500 dataset and 247 from the RSNA dataset—to form the second dataset 122. Under the guidance of doctors and radiologists, the selected CT slices were manually annotated with ground-truth segmentation masks for ICH lesions. The ground-truth bounding boxes were generated from the ground-truth segmentation masks using the mask_to_boxes algorithm from the PyTorch library. To maximize the segmentation accuracy, each ground-truth bounding box fully covered the ICH lesion and contained only one subtype of ICH lesion. The localized images 310 that form the second dataset 122 were extracted from the CT slices, wherein each localized image 310 is defined by the respective ground-truth bounding box.

In one example, the image segmentation model 120 includes the EM algorithm. As the EM algorithm is an unsupervised segmentation algorithm, no training was required. The second dataset 122 was thus used for evaluating the EM algorithm only.

In one example, the image segmentation model 120 includes the TransDeepLab model. Of the 347 localized CT slices in the second dataset 122, 279 slices were used for training, 32 slices for validation, and 36 slices for testing. Notably, the TransDeepLab model of the second stage was trained independently from the YOLOv5 model of the first stage. The TransDeepLab model was trained for 300 epochs with 0.01 learning rate and batch size 8 using a stochastic gradient descent (SGD) decaying optimizer.

The performance of the two-stage machine learning inference of the method 200 was then evaluated. The performance of the object detection model 110 was evaluated using the mean Average Precision computed at an Intersection over Union (IOU) threshold score of 0.5, also known as mAP@0.5. The performance of the image segmentation model 120 was evaluated using two segmentation metrics, namely the IOU score and the Dice coefficient, also known as the F1 score. The IOU score and the Dice coefficient were computed using the output segmentation masks 312 and the original ground-truth segmentation masks for each CT slice.

Four combinations of the two-stage approach of the method 200 were evaluated—(1) YOLOv5 model with EM algorithm; (2) YOLOv5 model and TransDeepLab model; (3) YOLOv5 model and Swin-Unet model; and (4) Faster R-CNN and EM algorithm. These are then compared with a ground-truth two-stage approach that was performed using the ground-truth bounding boxes and the TransDeepLab model.

The two-stage approach was also compared to some single-stage inference models. The single-stage inference models include no-new-Net (nnU-Net), DUCK-Net, SegResNet, Swin-Unet, U-Net, Feature Pyramid Network (FPN), EM algorithm alone, TransDeepLab model alone, and a YOLOv5-Seg model. nnU-Net, DUCK-Net, SegResNet, Swin-Unet, U-Net, and FPN are deep, supervised segmentation network architectures, whereas EM is an unsupervised algorithm. These single-stage models were trained with 300 epochs on the same second dataset 122 as for the TransDeepLab model. The original training configurations, such as optimizer and loss function, of the single-stage models were followed, except that the number of parameters was reduced to be closer to the two-stage models. For example, nnU-Net was trained with its default data augmentation such as foreground oversampling, but the base number of features was reduced to 24. For the other models such as DUCK-Net, SegResNet, and SwinUNet, only rotation and flipping were performed as data augmentation.

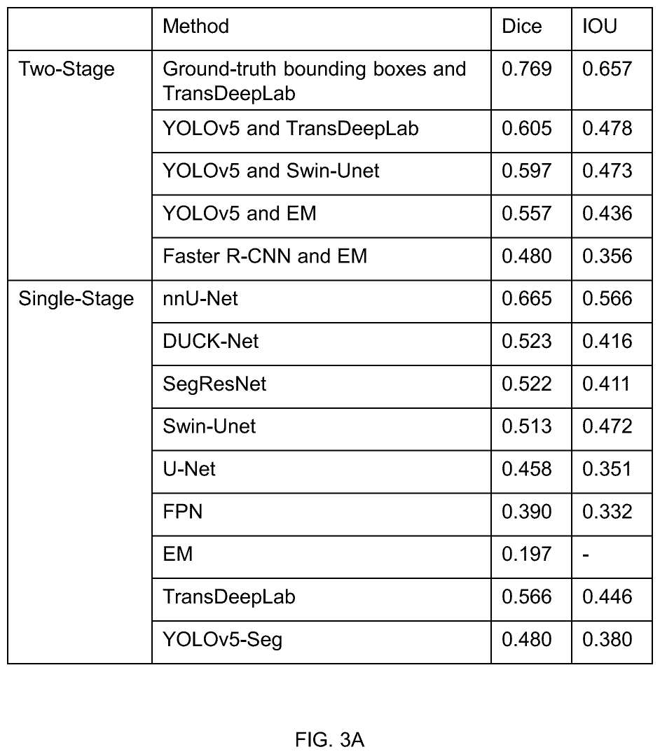

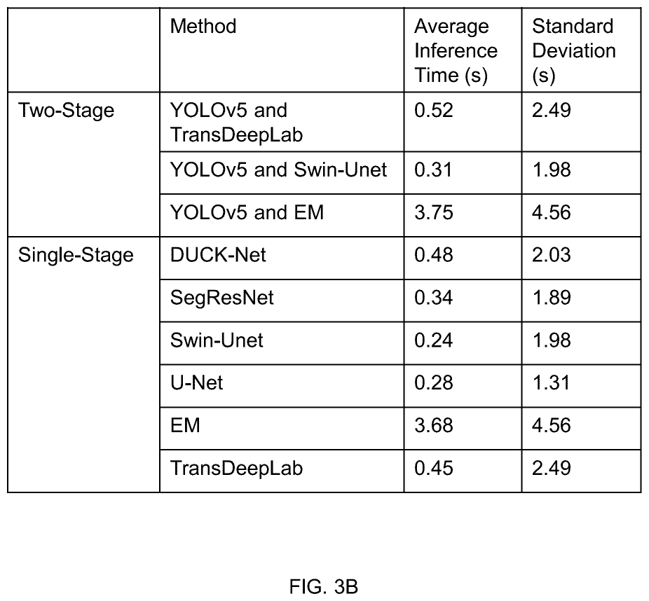

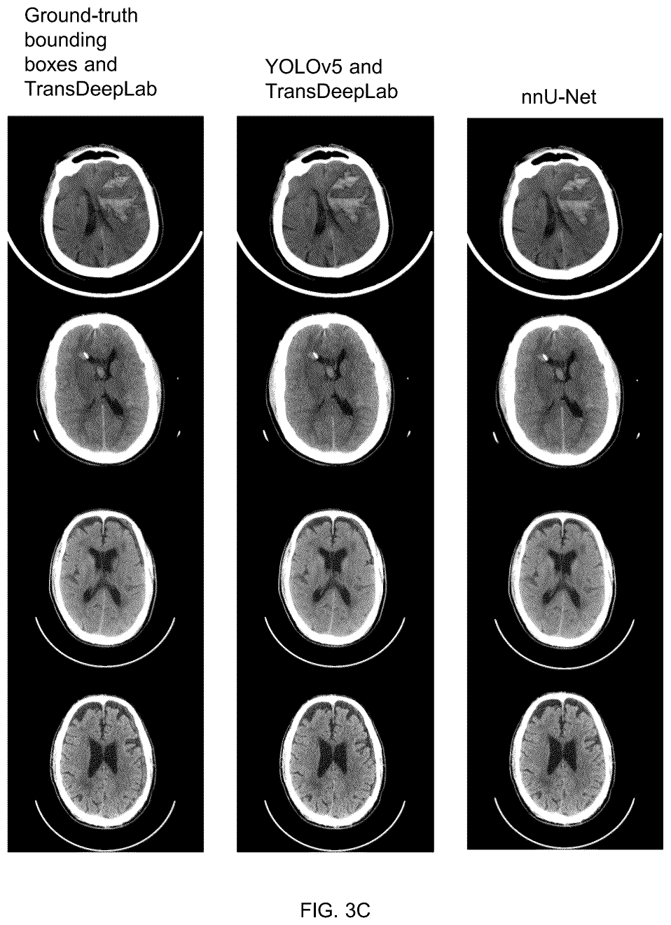

The performance evaluation results of the various two-stage and single-stage models are quantitatively shown in FIG. 3A and FIG. 3B. It is evident that directly using an unsupervised approach such the EM algorithm alone produces very low Dice coefficients. However, when the EM algorithm was used with an object detection model 110 such as YOLOv5 or Faster R-CNN, the Dice coefficients increased and outperformed some supervised single-stage models such as U-Net and FPN. When the image segmentation model 120 of the second stage was replaced by supervised models such as TransDeepLab or Swin-Unet, the Dice coefficients increased further, reaching 0.605 for the YOLOv5 and TransDeepLab combination.

The two-stage combinations performed better than single-stage models such as U-Net and SegResNet. This improvement in performance is likely due to the availability of larger datasets to train the object detection model 110 in the first stage, which proposed the ROIs 302 to alleviate the difficulty of training a single end-to-end segmentation model.

The YOLOv5 and TransDeepLab combination was the best-performing two-stage method in terms of both evaluation metrics compared to the ground-truth two-stage method (ground-truth bounding boxes and the TransDeepLab model) which achieved a Dice coefficient of 0.769 and an IOU of 0.657. A confusion matrix between the ground-truth two-stage method and the single-stage nnU-Net was analysed, and it was found that the improvement over nnU-Net, which had a Dice coefficient of 0.665 and an IOU of 0.566, was driven primarily by higher true positives and lower false negatives. Additionally, further experiments done to compare these two models in a 5-fold cross validation setting revealed that the improvement is statistically significant, with a p-value of 2.3×10⁻⁴ (two-sample t-test).

FIG. 3C shows visualization results of segmentation masks generated from the two-stage YOLOv5 and TransDeepLab models, and single-stage nnU-Net model, compared to the ground-truth two-stage method (ground-truth bounding boxes and the TransDeepLab model). It can be seen from the visualization results that the two-stage method is less prone to missing out on lesions.

The results shown in FIG. 3A to FIG. 3C demonstrate the superiority of the two-stage segmentation method 200 over single-stage methods. These results provide empirical evidence of the advantages of using bounding boxes 304 from the object detection model 110 to narrow the field of view for the image segmentation model 120, helping it to improve segmentation performance.

As the YOLOv5 and TransDeepLab combination was the best-performing two-stage combination, an ablation study was done to better understand the contributions of each stage. This combination and the round-truth combination were compared against the single TransDeepLab model and the single YOLOv5-Seg model. The single TransDeepLab model was used for direct image segmentation and was trained to segment ICH lesions regardless of the lesion subtype and without any bounding box guidance, since there is no first stage or object detection model 110 to provide the bounding boxes 304 nor any ground-truth bounding boxes. The single TransDeepLab model was trained for 600 epochs with the SGD decaying optimizer.

The YOLOv5-Seg model is a variant of the YOLOv5 model that was developed by training a segmentation head using the body of the trained YOLOv5 model. A combination of 4 convolutional layers, 3 up-sampling layers and 2 Bottleneck Cross Stage Partial modules were used to up-sample the latent vector to the segmentation mask. As compared to the current implementation of the segmentation head using the Proto module, the YOLOv5-Seg model had more layers and implemented two blocks, each containing a convolutional layer, an up-sampling layer, and the YOLO C3 dense block. Between these two blocks were two convolutional layers and an up-sampling layer. The YOLOv5-Seg model was trained on the union of the training and validation datasets until convergence by early-stopping, with batch size of 3 and stepped learning rate from 0.001.

Referring to the ablation study results in the last two rows of FIG. 3A, it is evident that the single TransDeepLab model performed better than the YOLOv5-Seg model, possibly suggesting that the second stage has a larger contribution to the overall performance of the method 200. When the YOLOv5 model was used to provide bounding boxes 304 for the TransDeepLab model, i.e. the two-stage method 200, the field of view exposed to the TransDeepLab model was narrowed and the segmentation performance (Dice coefficient) improved. When ground-truth bounding boxes were used, the Dice coefficient improved by over 30% compared to the single TransDeepLab model. The segmentation performance of the two-stage method 200 is clearly superior over single-stage methods, even though the single TransDeepLab model was trained for twice as many epochs as the TransDeepLab model in the two-stage combination.

Another key insight from the ablation study is the large increase in performance when using ground-truth bounding boxes instead of those predicted by the YOLOv5 model. The YOLOv5 model achieved an mAP@0.5 of 0.974 and an mAP@0.5:0.95 of 0.794 on the test dataset. There is some room for improvement in terms of lesion detection by the YOLOv5 model, and the results obtained from using the ground-truth bounding boxes shows the best results that could be obtained by the two-stage method 200. If the two-stage method 200 were to be implemented in clinical settings, neuroradiologists could make simple adjustments of the bounding box coordinates as a much quicker way of improving the Dice coefficients, as compared to manually editing the segmentation masks. These adjustments also create improved labels that can be used for fine-tuning the object detection model 110 in the first stage and consequentially improve segmentation performance in the second stage, eventuating to the high Dice coefficient of 0.769 from the ground-truth method.

As ICH is an emergency, it is important to ensure that the time taken to perform machine learning inference is not too long. The time taken to perform an inference of each CT slice in the test dataset was recorded for various two-stage and single-stage combinations. The average inference time and standard deviation for each combination are shown in FIG. 3B. It is evident that most of the tested combinations can perform segmentation in around 0.3 to 0.5 seconds. This amounts to approximately 15 seconds for a typical head CT scan with 30 slices. Further, two-stage combinations are not significantly slower than single-stage models. This can be attributed to the highly optimized implementation of the YOLOv5 model which allowed it to perform detection 5 to 10 times quicker—about 0.07 seconds each—than the time taken for segmentation.

The two-stage methods 200 with supervised second stages (TransDeepLab and Swin-Unet) have insignificant overheads as compared to single-stage models, making the two-stage methods 200 suitable for clinical deployment. Although the two-stage method 200 with unsupervised segmentation (EM) was slower than several single-stage models, making it less feasible for clinical deployment in emergencies. However, it could still find applications in other less time-sensitive use cases and/or where segmentation masks are not available at all, since the EM algorithm does not require training.

The two-stage method 200 leverages on large datasets of bounding box annotations to improve downstream segmentation performance. This is especially useful considering how datasets of segmentation masks are typically very small, and datasets of bounding boxes are more prevalent as manual annotation of bounding boxes is easier and quicker as it only involves identifying their coordinates. An ICH is a life-threatening condition, which could result in death if not treated expeditiously, automated ICH detection and segmentation using the method 200 would allow for faster ICH diagnosis, accelerate the process of triage, and allow doctors to rapidly treat ICH patients. Even if the output segmentation mask 312 is slightly inaccurate, it can still provide doctors with a good starting point for their examination of a patient's CT scan. This is because the automated method 200 reduces the need for radiologists to search tens or hundreds of thinly cut CT slices in each scan before making a diagnosis. The time and effort saved is especially significant in cases of multi-person accidents such as traffic accidents. In such situations, there may be too few CT scanners available, or enough radiologists present to diagnose the patients simultaneously. Hence, the system 100 and method 200 can be implemented in hospitals and clinics as it speeds up ICH diagnosis, thereby increasing the probability of survival for ICH patients and lowering the risk of brain damage.

In addition, the system 100 and method 200 could also provide the valuable data insights like lesion volume estimation, survival analysis, and disease prognosis. Furthermore, the system 100 and method 200 would be useful in medical institutions located at sites that lack expertise resources. For example, the system 100 and method 200 can aid junior or trainee radiologists in their work by increasing the sensitivity of ICH detection and reducing misinterpretation of some common ICH subtypes, thereby mitigating the consequences of misdiagnoses.

Although the system 100 and method 200 are described in various embodiments herein for diagnosis of ICH lesions, it will be appreciated that the system 100 and method 200 can be applied for medical image segmentation of other types of lesions.

In the foregoing detailed description, embodiments of the present disclosure in relation to a computer system and a computerized method for segmenting medical images are described with reference to the provided figures. The description of the various embodiments herein is not intended to call out or be limited only to specific or particular representations of the present disclosure, but merely to illustrate non-limiting examples of the present disclosure. The present disclosure serves to address at least one of the mentioned problems and issues associated with the prior art. Although only some embodiments of the present disclosure are disclosed herein, it will be apparent to a person having ordinary skill in the art in view of this disclosure that a variety of changes and/or modifications can be made to the disclosed embodiments without departing from the scope of the present disclosure. Therefore, the scope of the disclosure as well as the scope of the following claims is not limited to embodiments described herein.

1. A computerized method for segmenting a medical image of an organ, the method comprising:
detecting, using an object detection model, a region of interest in the medical image, the region of interest comprising a lesion in the organ;
demarcating, using the object detection model, a bounding box around the region of interest;
extracting a localized image comprising the region of interest from the medical image, the localized image defined by the bounding box;
segmenting, using an image segmentation model that is independent from the object detection model, the localized image comprising the region of interest;
predicting, using the image segmentation model, a segmentation mask of the lesion in the localized image; and
outputting the segmentation mask from the localized image to the medical image to facilitate medical diagnosis of the lesion,
wherein the object detection model is trained using a first dataset of training images comprising medical images of the organ, each medical image in the first dataset comprising a ground-truth bounding box for one or more lesions in the organ.

2. The method according to claim 1, comprising:
detecting, using the object detection model, a plurality of regions of interest in the medical image, each region of interest comprising a lesion in the organ;
demarcating, using the object detection model, the bounding box around the plurality of regions of interest;
extracting the localized image comprising the plurality of regions of interest from the medical image;
segmenting, using the image segmentation model, the localized image comprising the plurality of regions of interest;
generating, using the image segmentation model, segmentation masks of the respective lesions in the localized image; and
outputting the segmentation masks from the localized image to the medical image to facilitate medical diagnosis of the lesions.

3. The method according to claim 1, wherein the trained object detection model comprises a YOLOv5 model.

4. The method according to claim 3, wherein hyperparameters of the YOLOv5 model are optimized using a genetic algorithm.

5. The method according to claim 1, wherein the image segmentation model is independently trained using a second dataset of training images comprising localized images of lesions in the organ, each localized image in the second dataset comprising one or more ground-truth segmentation masks for one or more lesions in the organ.

6. The method according to claim 5, wherein the trained image segmentation model comprises a TransDeepLab model.

7. The method according to claim 1, wherein the image segmentation model comprises an untrained Expectation-Maximization algorithm.

8. The method according to claim 1, further comprising pre-processing the medical image before detecting the region of interest.

9. The method according to claim 8, wherein said pre-processing of the medical image comprises windowing the medical image and/or removing regions of skull tissue and calcification in the medical image.

10. The method according to claim 1, wherein the organ is a brain, and the lesion is an intracranial haemorrhage.

11. A non-transitory computer-readable medium having stored thereon instructions that, when executed, cause a processor to perform the computerized method according to claim 1.

12. A computer system for segmenting a medical image of an organ, the system comprising:
an object detection model that is trained using a first dataset of training images comprising medical images of the organ, each medical image in the first dataset comprising a ground-truth bounding box for one or more lesions in the organ;
an image segmentation model that is independent from the object detection model; and
a processor configured for:
detecting, using the object detection model, a region of interest in the medical image, the region of interest comprising a lesion in the organ;
demarcating, using the object detection model, a bounding box around the region of interest;
extracting a localized image comprising the region of interest from the medical image, the localized image defined by the bounding box;
segmenting, using the image segmentation model, the localized image comprising the region of interest;
predicting, using the image segmentation model, a segmentation mask of the lesion in the localized image; and
outputting the segmentation mask from the localized image to the medical image to facilitate medical diagnosis of the lesion.

13. The system according to claim 12, wherein the processor is configured for:
detecting, using the object detection model, a plurality of regions of interest in the medical image, each region of interest comprising a lesion in the organ;
demarcating, using the object detection model, the bounding box around the plurality of regions of interest;
extracting the localized image comprising the plurality of regions of interest from the medical image;
segmenting, using the image segmentation model, the localized image comprising the plurality of regions of interest;
generating, using the image segmentation model, segmentation masks of the respective lesions in the localized image; and
outputting the segmentation masks from the localized image to the medical image to facilitate medical diagnosis of the lesions.

14. The system according to claim 12, wherein the trained object detection model comprises a YOLOv5 model.

15. The system according to claim 14, wherein parameters of the YOLOv5 model are optimized using a genetic algorithm.

16. The system according to claim 12, wherein the image segmentation model is independently trained using a second dataset of localized images of lesions in the organ, each localized image in the second dataset comprising one or more ground-truth segmentation masks for one or more lesions in the organ.

17. The system according to claim 16, wherein the trained image segmentation model comprises a TransDeepLab model.

18. The system according to claim 12, wherein the image segmentation model comprises an untrained Expectation-Maximization algorithm.

19. The system according to claim 12, wherein the processor is configured for pre-processing the medical image before detecting the region of interest.

20. The system according to claim 19, wherein said pre-processing of the medical image comprises windowing the medical image and/or removing regions of skull tissue and calcification in the medical image.
